(19) United States
(12) Patent Application Publication
(10) Pub. No.: US 2026/0272362 A1
(43) Pub. Date: Sep. 17, 2026

(54) INFORMATION PROCESSING DEVICE, STORAGE MEDIUM, AND INFORMATION PROCESSING METHOD

(71) Applicant: TOYOTA BOSHOKU KABUSHIKI KAISHA, Aichi (JP)

(72) Inventor: Shin INAMI, Aichi (JP)

(73) Assignee: TOYOTA BOSHOKU KABUSHIKI KAISHA, Aichi (JP)

(21) Appl. No.: 19/560,667

(22) Filed: Mar. 9, 2026

(30) Foreign Application Priority Data
Mar. 11, 2025 (JP) 2025-038498

(51) Int. Cl. A61B 5/372; A61B 5/00; A61B 5/377

(52) U.S. Cl. CPC A61B 5/372; A61B 5/377; A61B 5/7264

(57) ABSTRACT

An information processing device includes: an obtaining section that obtains electroencephalogram data indicative of an electroencephalogram of a user; a first estimation section that estimates, by using a model for estimating an activity state of a brain network, the activity state of the brain network from the electroencephalogram data; and a second estimation section that estimates whether or not a thought state of the user is a deliberation state, on the basis of a probability that the activity state of the brain network switches.

This Nonprovisional application claims priority under 35 U.S.C. § 119 on Patent Application No. 2025-038498 filed in Japan on Mar. 11, 2025, the entire contents of which are hereby incorporated by reference.

### TECHNICAL FIELD

The present disclosure relates to an information processing device, a storage medium, and an information processing method.

### BACKGROUND ART

Conventionally, various technologies using measurement data of biological information (particularly electroencephalograms) have been developed (for example, Patent Literatures 1 to 5). Patent Literature 1 discloses cognitive emotional training that “is also associated with changes in short-term plasticity of brain networks”. Patent Literature 2 discloses a technique that “allows relatively quick estimation of activities of various brain networks with use of signals of brain waves measured by a relatively simplified method such as EEG measurement”. Patent Literature 3 discloses a technique for “determining a brain state corresponding to each brain activity pattern on the basis of a degree of a spatial correlation between (a) each of a plurality of brain activity patterns and (b) a plurality of templates respectively indicating a plurality of predetermined brain states”. Patent Literature 4 discloses a technique for “guiding a human thought process to a contemplation state in which the human thinks deeply about things”. Patent Literature 5 discloses, as a technique for analyzing functional activity data of a brain, a technique for “obtaining functional activity data for a region of a brain of a first individual, wherein the functional activity data represents a collective activity of a plurality of neurons in the region; and estimating relative activities of neural pathways regulated by a plurality of neuronal subtypes by computationally processing the functional activity data”.

### CITATION LIST

### Patent Literatures

### Patent Literature 1

Japanese Patent Application Publication, Tokukai, No. 2023-159206

### Patent Literature 2

Japanese Patent Application Publication, Tokukai, No. 2019-93008

### Patent Literature 3

Japanese Patent Application Publication, Tokukai, No. 2024-40721

### Patent Literature 4

Japanese Patent Application Publication, Tokukai, No. 2024-80008

### Patent Literature 5

Japanese Patent Application Publication, Tokukai, No. 2019-508109

### SUMMARY OF INVENTION

### Technical Problem

In recent years, with the spread of smartphones and the Internet, use of social networking service (SNS), artificial intelligence (AI), and the like is becoming a part of daily life. SNS, AI, and the like are convenient. At the same time, however, various risks are of concern regarding SNS, AI, etc. For example, the problems have been reported. That is, there is a negative correlation between use of SNS and academic performance, and human attention span decreases year by year. Moreover, due to the development of AI, people may become dependent on AI for selecting and scrutinizing information; furthermore, as people are deprived of opportunities for deliberation by AI-induced filter bubbles, they may get used to using only intuition and become unable to activate deliberation. In view of this, there has been a demand for development of a system that provides a stimulus to trigger activation of deliberation. However, it is first necessary to acknowledge whether a thought state is a deliberation state or an intuition state. However, none of Patent Literatures 1 to 5 discloses anything about this point.

An aspect of the present disclosure has an object to estimate whether a thought state is a deliberation state.

### Solution to Problem

In order to attain the above object, an information processing device in accordance with an aspect of the present disclosure includes: an obtaining section that obtains (a) electroencephalogram data indicative of an electroencephalogram of a user or (b) biological information substitutable for the electroencephalogram; a first estimation section that estimates, by using a model for estimating an activity state of a brain network, the activity state of the brain network from the electroencephalogram data or the biological information; and a second estimation section that estimates whether or not a thought state of the user is a deliberation state, on a basis of a probability that the activity state of the brain network estimated by the first estimation section switches.

In order to attain the above object, an information processing method in accordance with an aspect of the present disclosure includes: an obtaining step of obtaining (a) electroencephalogram data indicative of an electroencephalogram of a user or (b) biological information substitutable for the electroencephalogram; a first estimation step of estimating, by using a model for estimating an activity state of a brain network, the activity state of the brain network from the electroencephalogram data or the biological information; and a second estimation step of estimating whether or not a thought state of the user is a deliberation state, on a basis of a probability that the activity state of the brain network estimated in the first estimation step switches.

An information processing device in accordance with each aspect of the present disclosure may be realized by a computer. In this case, the present disclosure encompasses: an information processing program causing a computer to operate as each section (software element) included in the information processing device so as to realize the information processing device as the computer; and a computer-readable storage medium in which the information processing program is stored.

### Advantageous Effects of Invention

In accordance with an aspect of the present disclosure, it is possible to estimate whether a thought state is a deliberation state.

### BRIEF DESCRIPTION OF DRAWINGS

FIG. 1 is a view illustrating an example of a scene to which an information processing system in accordance with a first embodiment is applied.

FIG. 2 is a view illustrating an example of a configuration of the information processing system in accordance with the first embodiment.

FIG. 3 is a block diagram illustrating an example of a hardware configuration of the information processing device in accordance with the first embodiment.

FIG. 4 is a block diagram illustrating an example of functions of a processor of the information processing device in accordance with the first embodiment.

FIG. 5 is a view for explaining a default mode network, a salience network, and an executive network, each of which is an activity state of a brain network.

FIG. 6 is a graph illustrating an example of an analysis result of electroencephalogram data.

FIG. 7 is a graph illustrating an example of an analysis result of electroencephalogram data.

FIG. 8 is a view for explaining PVT.

FIG. 9 is a view for explaining WCST.

FIG. 10 is a view for explaining probability of an activity state of a brain network switching per unit time.

FIG. 11 is a flowchart illustrating an example of a process executed by the information processing device in accordance with the first embodiment.

FIG. 12 is a view illustrating an example of a configuration of an information processing system in accordance with a second embodiment.

FIG. 13 is a block diagram illustrating an example of functions of a processor of the information processing device in accordance with the second embodiment.

FIG. 14 is a flowchart illustrating an example of a process executed by the information processing device in accordance with the second embodiment.

### DESCRIPTION OF EMBODIMENTS

### First Embodiment

The following will describe details of a first embodiment of the present disclosure with reference to the drawings. In the description of the drawings, the same reference signs are assigned to features having the same or substantially same functions, and the descriptions thereof are omitted.

Notations such as “first” and “second” in the present disclosure are used to distinguish one component from another, and are not intended to limit the number, order, priority, and/or the like of the components. For example, a description including a “first element” and a “second element” does not mean that there are only two elements, i.e., the “first element” and the “second element”, nor does it mean that the “first element” must precede the “second element”.

Furthermore, unless otherwise specified, the phrase “on the basis of” as used herein does not mean “only on the basis of”. The phrase “on the basis of” means both “only on the basis of” and “at least on the basis of”.

### Application Example of Information Processing System

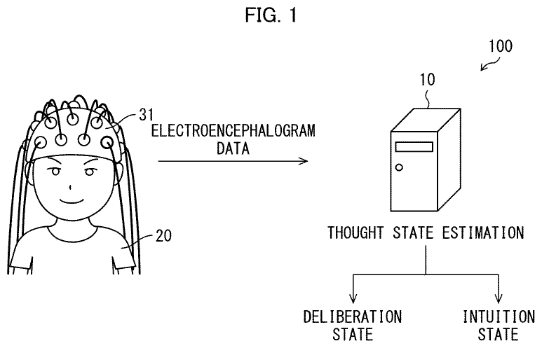

First, an application example of an information processing system 100 of the present embodiment will be described with reference to FIG. 1. FIG. 1 is a view illustrating an example of a scene to which the information processing system 100 is applied. FIG. 1 illustrates a case where the information processing system 100 estimates, on the basis of electroencephalogram data of the user 20, whether a thought state of a user 20 is a deliberation state or an intuition state. The information processing system 100 includes: a device that obtains biological information of the user 20 (i. e., an electroencephalogram sensor 31 that measures an electroencephalogram in FIG. 1); and an information processing device 10 that analyzes the electroencephalogram data measured by the electroencephalogram sensor 31.

As shown in FIG. 1, the electroencephalogram sensor 31 is attached to the head of the user 20. The electroencephalogram sensor 31 measures an electroencephalogram of the user 20. Electroencephalogram data indicative of the electroencephalogram measured by the electroencephalogram sensor 31 is transmitted to the information processing device 10 by a predetermined communication method.

The information processing device 10 estimates the thought state of the user 20 on the basis of the electroencephalogram data. Specifically, the information processing device 10 estimates, on the basis of the electroencephalogram data, whether the thought state of the user 20 is the deliberation state or the intuition state.

### Deliberation State

The definition of the “deliberation state” explained in the present embodiment is not particularly limited. However, as an example, the deliberation state may be defined as a state of thinking deeply about things and acting, judging, or the like cautiously after taking into consideration a wide variety of viewpoints and information.

### Intuition State

The definition of the “intuition state” explained in the present embodiment is not particularly limited. However, as an example, the intuition state may be defined as a state of acting, judging, or the like instantaneously without thinking deeply about things.

In the present embodiment, the thought state of the user 20 is described as selected from among two alternatives, i.e., the deliberation state and the intuition state. That is, when the thought state which is in the deliberation state switches, this means that the thought state switches to the intuition state. Similarly, when the thought state which is in the intuition state switches, this means that the thought state switches to the deliberation state. Of course, the thought state is not limited to the two states, i.e., the deliberation state and the intuition state. Alternatives of the thought state may include not only the deliberation state and the intuition state but also a third state, a fourth state, and the like.

Needless to say, neither the deliberation state nor the intuition state refers to a property indicating superiority or inferiority. Rather, it is important to use the deliberation state and the intuition state selectively according to the situation. However, as described above, the development of AI in recent years may lead to the following problem. That is, as AI carries out deliberation on behalf of people, people may get used to using only intuition and become unable to activate deliberation.

In order to solve such a problem, there has been a demand for development of a system that provides a stimulus to trigger activation of deliberation. For this purpose, it is first necessary to acknowledge whether the thought state is the deliberation state or the intuition state. Therefore, the present embodiment employs a configuration that estimates, by using electroencephalogram data, whether the thought state of the user 20 is the deliberation state or the intuition state. This configuration makes it possible to estimate the thought state of the user 20 in a non-invasive manner. The details will be described below.

### Configuration Example of Information Processing System 100

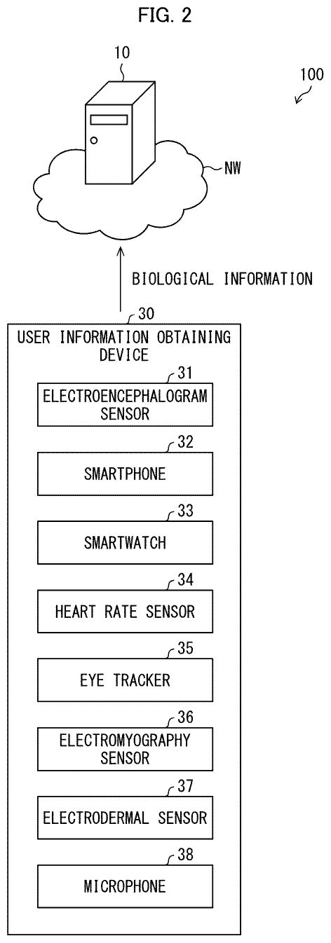

The following will describe, with reference to FIG. 2, an example of a configuration of the information processing system 100 in accordance with the present embodiment. As shown in FIG. 2, the information processing system 100 includes an information processing device 10 and a user information obtaining device 30. The information processing device 10 is communicably connected to the user information obtaining device 30 via a communication network NW. There is no particular limitation on the communication network NW. For example, the communication network NW may be the Internet.

### User Information Obtaining Device 30

As described with reference to FIG. 1, the user information obtaining device 30 includes, for example, the electroencephalogram sensor 31 that measures an electroencephalogram of the user 20. According to the method described as an example in the present embodiment, the electroencephalogram data of the user 20 is analyzed so as to estimate whether the thought state of the user 20 is the deliberation state or the intuition state. The biological information used therefor is not limited to the electroencephalogram, and may be biological information substitutable for the electroencephalogram. Examples of the biological information substitutable for the electroencephalogram include a respiration rate, a heart rate, a blood pressure, and the like. The respiration rate can be analyzed, for example, from face image data of the user 20 captured by a smartphone 32 included in the user information obtaining device 30. Furthermore, the heart rate and blood pressure can be obtained, for example, by a smartwatch 33 worn on the wrist of the user 20.

As another example, the heart rate may be obtained by a heart rate sensor 34. Furthermore, the biological information substitutable for the electroencephalogram may include, in addition to the above information, an eye gaze movement, a muscle activity level, electrodermal activity (EDA), voice data, and the like. In order to obtain these pieces of biological information, the user information obtaining device 30 may include an eye tracker 35, an electromyography (EMG) sensor 36, an electrodermal sensor 37, and a microphone 38. The eye tracker 35 obtains data indicative of eye gaze movement of the user 20. The electromyography sensor 36 obtains data indicative of an activity level of a muscle of the user 20. The electrodermal sensor 37 obtains data indicative of an electrodermal activity of the user 20. The microphone 38 obtains voice data of the user 20.

The electroencephalogram sensor 31 included in the user information obtaining device 30 measures the electroencephalogram data of the user 20 in the above-described manner. Furthermore, the smartphone 32, the smartwatch 33, the heart rate sensor 34, the eye tracker 35, the electromyography sensor 36, the electrodermal sensor 37, and the microphone 38 included in the user information obtaining device 30 obtain the biological information substitutable for the electroencephalogram. The electroencephalogram data or the biological information substitutable for the electroencephalogram obtained by the user information obtaining device 30 is transmitted to the information processing device 10.

### Information Processing Device 10

The information processing device 10 obtains the electroencephalogram data or the biological information substitutable for the electroencephalogram from the user information obtaining device 30 via the communication network NW. The information processing device 10 estimates, on the basis of the electroencephalogram data or the biological information substitutable for the electroencephalogram, whether the thought state of the user 20 is the deliberation state or the intuition state.

There is no particular limitation on a location where the information processing device 10 is installed. For example, the information processing device 10 may be installed in a management center of a business operator that provides a thought state estimation service which is described in the present embodiment. The information processing device 10 may be installed in Japan or outside Japan. The information processing device 10 may be configured as a physical server or as a cloud server.

### Hardware Configuration of Information Processing Device 10

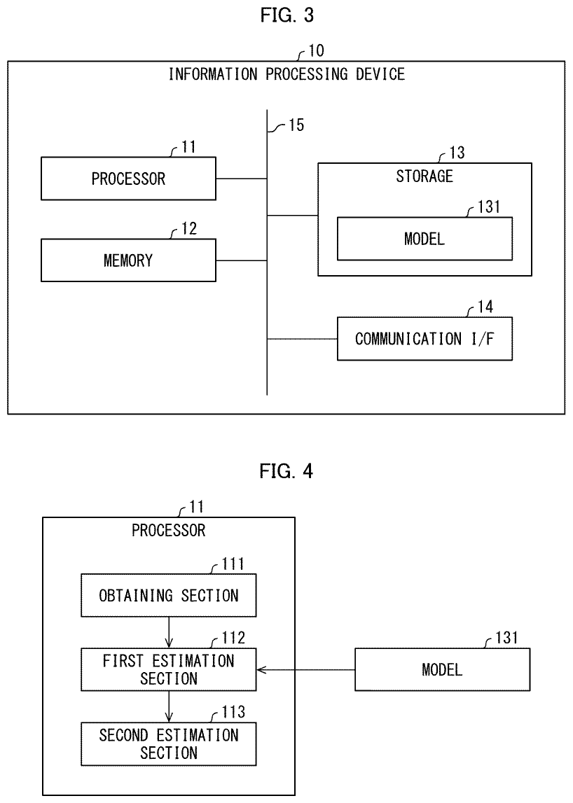

Next, the following will describe, with reference to FIG. 3, an example of a hardware configuration of the information processing device 10. FIG. 3 is a block diagram illustrating an example of the hardware configuration of the information processing device 10.

As shown in FIG. 3, the information processing device 10 includes a processor 11, a memory 12, a storage 13, and a communication interface (I/F) 14. These components are connected to each other via a bus 15 in such a manner that these components can mutually communicate with each other.

The processor 11 reads a program from the storage 13, loads the program into the memory 12, and executes a process according to the program. As such a program, a program that causes a computer to execute at least a part of the below-described functions is used. Note that the program may be one that exerts the functions in combination with another program already stored in the storage 13 or in combination with another program implemented in another device. Furthermore, the program may be delivered to the information processing device 10 by wireless communication. In this case, the processor 11 will load the delivered program into the memory 12 and execute the process. That is, the program does not necessarily have to be stored in the storage 13.

The processor 11 is not particularly limited, but is realized as, for example, a central processing unit (CPU), a graphics processing unit (GPU), a microprocessor unit (MPU), a field-programmable gate array (FPGA), or the like. In FIG. 3, a single processor is illustrated. However, this is not limitative. Alternatively, a plurality of processors may be provided.

The memory 12 is a computer-readable storage medium and is configured by at least one selected from the group consisting of, for example, a random access memory (RAM), a read only memory (ROM), an erasable programmable ROM (EPROM), an electrically erasable programmable ROM (EEPROM) (registered trademark), and the like. Note that such a memory 12 may also be referred to as a register, a cache, a main memory (main storage device), or the like.

The storage 13 is a computer-readable storage medium and stores various data therein. Such a storage 13 is configured by, for example, a hard disk drive (HDD), a solid state drive (SSD), or the like. Note that the storage 13 may be a portable storage medium such as a flexible disk, an optical disc, a compact disc, or a Blu-ray (registered trademark) disc. Furthermore, the storage 13 may also be referred to as an auxiliary storage device.

The storage 13 in the present embodiment stores a model 131 for estimating an activity state of a brain network. Details of the model 131 will be described later. The model 131 includes a machine learning model, a template model, a regression model, and the like.

The communication I/F 14 is an interface for communicating with an external device. The communication I/F 14 is implemented as, for example, hardware such as a network adapter, various types of communication software, or a combination thereof, and is configured to provide wireless communication or wired communication via a communication network.

Note that the hardware configuration of the information processing device 10 is not limited to the configuration described above. For example, the information processing device 10 may include an input-output I/F as hardware. Such an input-output I/F may include, for example, an information input device such as a keyboard or a pointing device (e.g., a mouse, a touch panel), a voice input device such as a microphone, and an image input device such as a camera. Furthermore, the input-output I/F may include an image output device such as a display and a voice output device such as a speaker.

### Functional Example of Processor 11

Next, the following will describe, with reference to FIG. 4, an example of functions of the processor 11 of the information processing device 10. FIG. 4 is a block diagram illustrating an example of the functions of the processor 11. The processor 11 reads a program from the storage 13 and executes the program using the memory 12 as a work area, thereby functioning as an obtaining section 111, a first estimation section 112, and a second estimation section 113. An element described as a “. . . section” may be rephrased as a “. . . circuit”, a “. . . device” , or a “. . . apparatus” , or may be rephrased as a “. . . step”, a “. . . procedure” , or a “. . . process” . That is, an element described as a “. . . section” may be realized by a program stored in the storage 13 as described above. Alternatively, an element described as a “. . . section” may be implemented only by hardware such as an element, a device, a substrate, and a wiring or by a combination of software and hardware. These functions will be described below.

As described with reference to FIG. 2, the obtaining section 111 obtains, from the user information obtaining device 30, electroencephalogram data of the user 20 or biological information substitutable for the electroencephalogram. Hereinafter, unless otherwise specified, the biological information of the user 20 obtained from the user information obtaining device 30 will be described as “electroencephalogram data”.

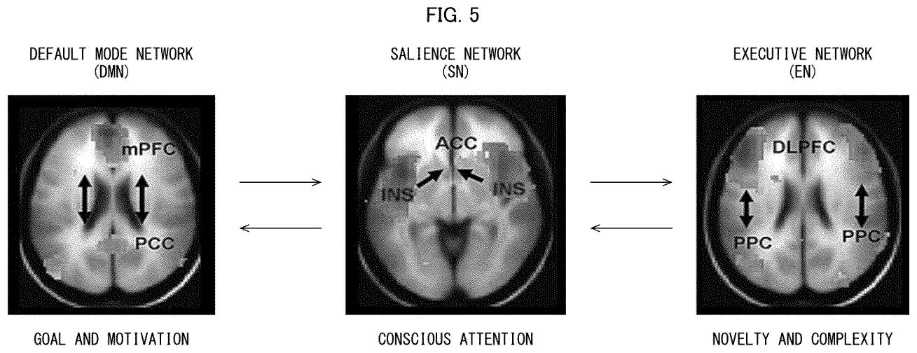

The first estimation section 112 estimates, by using the model 131 for estimating the activity state of the brain network, an activity state of a brain network from the electroencephalogram data obtained by the obtaining section 111. The following will describe an “activity state of a brain network” in the present embodiment. It is known that a brain network functions through mutual cooperation of different regions of the brain. Activity states of a brain network include three states, specifically, a default mode network (DMN), a salience network (SN), and an executive network (EN). Here, the default mode network, the salience network, and the executive network will be described with reference to FIG. 5.

### Default Mode Network

The default mode network is a network related to a goal and motivation, and is a network that is activated mainly while self-related thoughts and internal thoughts (e.g., recalling the past, planning for the future) are carried out. Main regions that are activated are, as shown in FIG. 5, a medial prefrontal cortex (mPFC), a posterior cingulate cortex (PCC), and the like.

### Salience Network

The salience network is a network related to conscious attention, and is a network that is activated mainly while attention is paid to an important external stimulus and emotional information. Main regions that are activated are, as shown in FIG. 5, an anterior cingulate cortex (ACC), an insular cortex (Ins), and the like.

### Executive Network

The executive network is a network related to novelty and complexity, and is network that is activated mainly while higher-order cognitive functions such as decision-making, problem-solving, planning, and self-control are carried out. Main regions that are activated are, as shown in FIG. 5, a dorsolateral prefrontal cortex (DLPFC), a posterior parietal cortex (PPC), and the like.

The activity states of the default mode network, the salience network, and the executive network described above switch due to various factors, including an external stimulus. On the basis of the electroencephalogram data of the user 20, the first estimation section 112 estimates which of these three states the activity state of the brain network of the user 20 is in.

### Example of Method for Estimating Activity State of Brain Network

The above description has explained that the model 131 includes the machine learning model, the template model, the regression model, and the like. The description here will discuss a case where the model 131 is a machine learning model. In this case, the first estimation section 112 estimates, by using the machine learning model, which of the three states (DMN, SN, EN) an activity state of the brain network of the user 20 is in. The machine learning model in the present embodiment is a trained model that has been trained in advance on a feature(s) of electroencephalogram data. There is no particular limitation on the training method. For example, the training method may be the one carried out in the following manner. First, data preprocessing is carried out before extraction of the feature(s) of the electroencephalogram data. The data preprocessing carries out noise removal such as deletion of data having a missing value and data transformation such as standardization and/or normalization. As the algorithm for extracting the features, a well-known algorithm may be used. Examples of the well-known algorithm include principal component analysis, Kalman filter, and Bayesian estimation.

The feature extraction process may be carried out in accordance with not only the above-described algorithm but also a perspective(s) such as intensities, degrees of power, and/or phases between channels, between time points, and respective frequency bands or a combination of these perspectives. As another example, the feature(s) may be extracted on the basis of a statistic obtained from a combination of (a) a basic statistic per data unit, such as a sum, a mean, a median, a variance, and/or a standard deviation, and (b) a data unit, such as a correlation coefficient, a distance, a coherence, an autocorrelation, a cross-correlation, and/or a linear approximation.

By using the extracted feature(s), supervised learning is carried out. As an algorithm for the machine learning, a well-known algorithm may be used. Examples of the well-known algorithm include decision tree, random forest, extra tree, gradient boosting decision tree, AdaBoost, support vector machine, Gaussian process regression, generalized linear modeling, L1 and L2 penalized regression, Bayesian ridge regression, k-nearest neighbors, and neural network.

The first estimation section 112 inputs the electroencephalogram data of the user 20 into the machine learning model to estimate which of the three states (DMN, SN, EN) the activity state of the brain network of the user 20 is in.

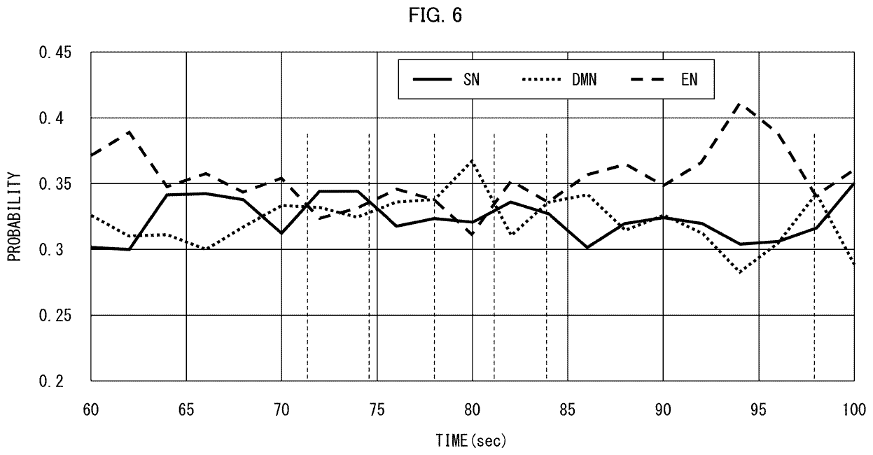

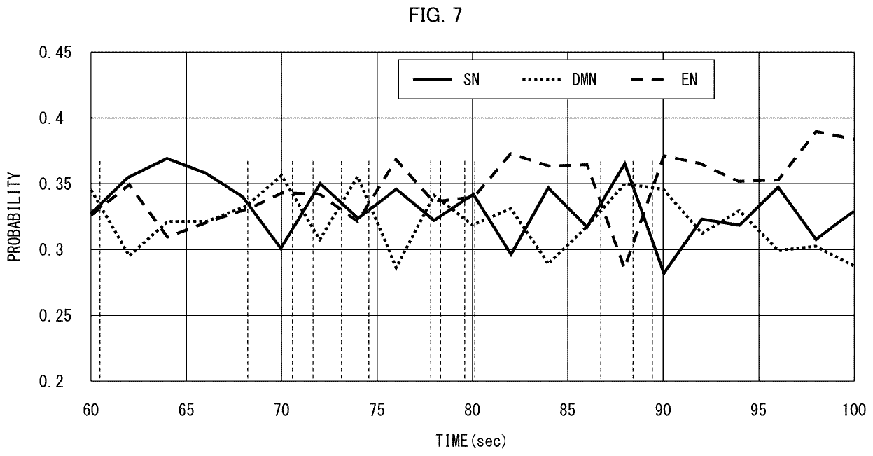

An example of an estimation result obtained by using the machine learning model is shown in FIGS. 6 and 7.

FIG. 6 is a graph obtained by inputting, into the machine learning model, electroencephalogram data measured when a certain subject (hereinafter, referred to as a “subject A”) carried out a psychomotor vigilance test (PVT). FIG. 7 is a graph obtained by inputting, into the machine learning model, electroencephalogram data measured when the subject A conducted a Wisconsin card sorting test (WCST). In FIGS. 6 and 7, “SN” denotes the salience network, “DMN” denotes the default mode network, and “EN” denotes the executive network.

### PVT and WCST

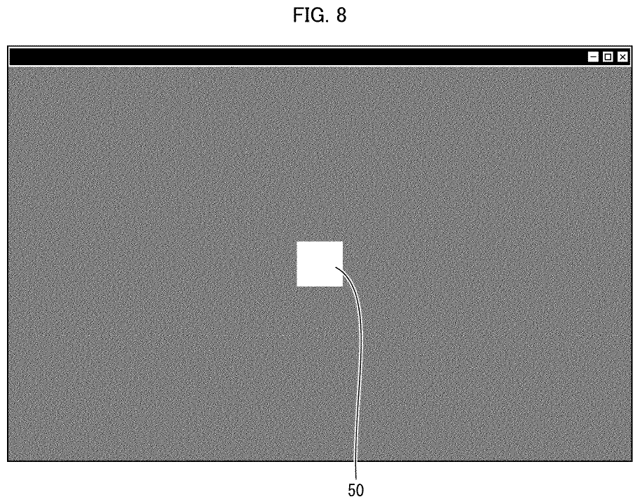

The description here will briefly explain PVT and WCST. The PVT is a test aimed at evaluating the state of attention and concentration by measuring a reaction time of a subject. In the PVT, as shown in FIG. 8, the subject is required to react as quickly as possible to a stimulus (e. g., a point of light) that appears randomly on a screen. The subject reacts to a stimulus by pressing a button 50 at a moment that the stimulus is displayed.

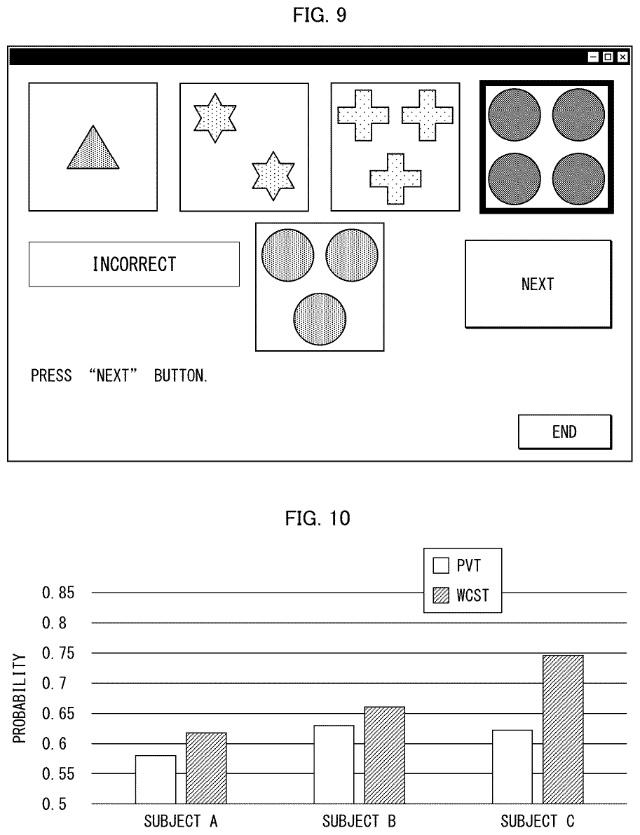

The WCST is a test aimed mainly at evaluating cognitive functions. In the WCST, as shown in FIG. 9, cards in which figures of different colors, shapes, and quantities are depicted are used. Typically, four types of cards are used. In each of these cards, a figure of a different color (e.g., red, blue, green, or yellow), a different shape (e.g., a circle, a triangle, a star, or a cross), and a different quantity (e.g., any of 1 to 4) is depicted. The subject is given the cards and instructed to sort them according to a specific criterion. Initially, the classification criterion for the cards is unknown, and thus the subject carries out the classification through trial and error. Each time the subject classifies a card, feedback is given as to whether it is correct or incorrect. If the classification is correct, the subject can proceed to a next card.

It can be expressed that the PVT is a test that requires intuition and the WCST is a test that requires deliberation.

In FIGS. 6 and 7, the horizontal axis indicates test time, and the vertical axis indicates a probability of an activity state of the brain network estimated by the machine learning model. For example, taking a point of 60 seconds in FIG. 6, it shows that a probability that the activity state of the brain network is the salience network is approximately 30%, a probability that the activity state of the brain network is the default mode network is approximately 32%, and a probability that the activity state of the brain network is the executive network is approximately 37%. Therefore, at the point of 60 seconds, it is estimated that the probability that the activity state of the brain network of the user 20 is the executive network is highest.

The inventors focused on the fact that a switching frequency of an activity state of a brain network differs between the PVT and the WCST conducted for the same subject. This point will be specifically described. The switching frequency of the activity state of the brain network can be determined by counting the number of times the brain network having a highest estimated probability switches.

In FIG. 6, at a point of approximately 71 seconds, the activity state switches from the executive network to the salience network. At a point just before a point of 75 seconds, the activity state switches from the salience network to the executive network. At a point of approximately 78 seconds, the activity state switches from the executive network to the default mode network. At a point of approximately 81 seconds, the activity state switches from the default mode network to the executive network. At a point of approximately 84 seconds, the activity state switches from the executive network to the default mode network. Then, immediately after that, the activity state switches from the default mode network to the executive network. At a point of approximately 98 seconds, the activity state switches from the executive network to the default mode network. Then, immediately after that, the activity state switches from the default mode network to the executive network.

As discussed above, in the estimation result shown in FIG. 6, a total of eight switches occur between the point of 60 seconds and a point of 100 seconds.

In contrast, in FIG. 7, at a point after the point of 60 seconds, the activity state switches from the default mode network to the salience network. At approximately the point of 68 seconds, the activity state switches from the salience network to the default mode network. At a point after a point of 70 seconds, the activity state switches from the default mode network to the executive network. At approximately the point of 71 seconds, the activity state switches from the executive network to the salience network. At a point of approximately 73 seconds, the activity state switches from the salience network to the default mode network. At a point just before 75 seconds, the activity state switches from the default mode network to the executive network. At a point of approximately 77 seconds, the activity state switches from the executive network to the default mode network. Thereafter, the activity state switches from the default mode network to the executive network. At a point just before a point of 80 seconds, the activity state switches from the executive network to the salience network. At a point just after the point of 80 seconds, the activity state switches from the salience network to the executive network. At a point of approximately 86 seconds, the activity state switches from the executive network to the salience network. At a point of approximately 88 seconds, the activity state switches from the salience network to the default mode network. At a point just before a point of 90 seconds, the activity state switches from the default mode network to the executive network.

Thus, in the estimation results shown in FIG. 7, a total of 13 switches occur between the point of 60 seconds and the point of 100 seconds.

As described above, comparison between the estimation result of the PVT (FIG. 6) and the estimation result of the WCST (FIG. 7) reveals that a switching frequency of the activity state of the brain network is higher for the WCST than for the PVT. As discussed above, the WCST is a test that requires more deliberation than the PVT. Therefore, it can be said that the higher switching frequency means a higher probability that the thought state of the user 20 is the deliberation state.

In the present embodiment, a probability of switching per unit time is used as an index indicating how “the switching frequency is high”. This point will be described with reference to FIG. 10. FIG. 10 shows a probability that an activity state of a brain network of each subject (a respective one of subjects A to C) switches per unit time. In FIG. 10, the unit time is “2 seconds”. Therefore, FIG. 10 specifically shows a probability that the activity state of the brain network switches every 2 seconds. For example, FIG. 10 shows that, in the PVT, an activity state of a brain network of the subject A switches every 2 seconds with a probability of approximately 58%. Meanwhile, FIG. 10 shows that, in the WCST, the activity state of the brain network switches every 2 seconds with a probability of approximately 62%. The same applies to the subjects B and C.

On the basis of the probability that the activity state of the brain network estimated by the first estimation section 112 switches per unit time, the second estimation section 113 estimates whether the thought state of the user 20 is the deliberation state or the intuition state. The “probability that the activity state of the brain network estimated by the first estimation section 112 switches per unit time” mentioned herein corresponds to the probability explained with reference to FIG. 10. There is no particular limitation on the estimation method carried out by the second estimation section 113. For example, a threshold may be set in advance, and if the probability explained with reference to FIG. 10 (the probability estimated by the machine learning model) is higher than the threshold, the thought state of the user 20 may be estimated to be the deliberation state. In light of the example shown in FIG. 10, the probability serving as the threshold may be set at 65%, for example. If the threshold is set at 65%, it would not be possible to estimate whether the thought state of the subject A is the deliberation state or the intuition state. In such a case, the unit time may be optimized. In FIG. 10, the unit time is set to “2 seconds”. Alternatively, the unit time may be adjusted to an arbitrary numerical value to search for a threshold based on which the thought state of all users or a majority of users is estimated as the deliberation state.

The above description has discussed the case where the method using the machine learning model is employed as an example of the method for estimating which of the three states (DMN, SN, EN) the activity state of the brain network of the user 20 is in. However, this is not limitative. As a method other than the machine learning model, a template model, a regression model, or the like may be used.

### Template Model

There is no particular limitation on the method for generating the template model. For example, the template model may be generated in the following manner. The data preprocessing such as noise removal is the same as for the machine learning model, which has been described above. The template model may be generated by dividing the scalp potential distribution, spectral intensity, phase information, and the like into clusters on the basis of the waveform after noise removal or the extracted feature(s) and by use of a clustering method such as k-means, hierarchical cluster analysis, DBSCAN, GMM, self-organizing map, affinity propagation, and/or spectral clustering.

The estimation of the probability of the activity state of the brain network using the template model may be carried out as follows. The measured electroencephalogram data is compared with each template. The comparison method may be, for example, as follows. That is, a distance between the measured electroencephalogram data and each template is measured. There is no particular limitation on the “distance” mentioned here. The “distance” may be, for example, a Euclidean distance, a standardized Euclidean distance, a Mahalanobis distance, a Manhattan distance, a Chebyshev distance, a Minkowski distance, or the like. As another example, a similarity between the measured electroencephalogram data and each template is measured. There is no particular limitation on the “similarity” mentioned here. The “similarity” may be, for example, a similarity given using the above distance, a cosine similarity, a correlation coefficient, a coefficient of determination, or a similarity given using linear, polynomial, sigmoid, RBF, Laplacian, chi-squared, and/or kernel.

The first estimation section 112 can estimate the probability of the activity state of the brain network on the basis of an index such as the distance and/or similarity described above.

### Regression Model

There is no limitation on the method for generating the regression model. For example, the regression model may be generated in the following manner. The regression model may be generated by extracting a feature(s) of the electroencephalogram data and carrying out regression and classification, while using a fully connected model such as a multilayer perceptron, a time-series model such as an RNN or LSTM, a convolutional model such as a CNN, or a model including attention such as a transformer.

The first estimation Section 112 can estimate the probability of the activity state of the brain network by using a regression model.

### Processing Flow

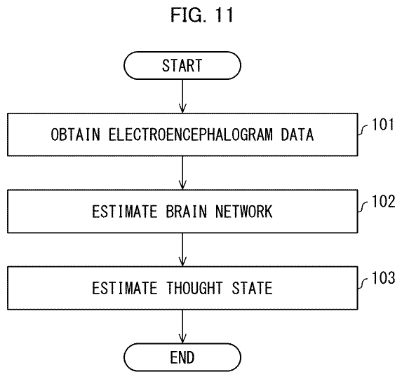

Next, the following will describe, with reference to FIG. 11, a flow of a process executed by the information processing device 10. FIG. 11 is a flowchart illustrating an example of the process executed by the information processing device 10. With reference to the flowchart, the following will describe the obtaining section 111, the first estimation section 112, and the second estimation section 113, which are functions of the information processing device 10.

In step S101, the obtaining section 111 obtains the electroencephalogram data of the user 20 from the electroencephalogram sensor 31, as described with reference to FIG. 2.

In step S102, by using the model 131 for estimating the activity state of the brain network, the first estimation section 112 estimates the activity state of the brain network on the basis of the electroencephalogram data obtained in the process carried out in step S101. The estimation results are output as indices of respective probabilities (i.e., a probability of being the default mode network, a probability of being the salience network, and a probability of being the executive network). The graphs plotting these probabilities over time are FIGS. 6 and 7 described above.

In step S103, on the basis of the probability that the activity state of the brain network estimated in the process of step S102 switches per unit time, the second estimation section 113 estimates whether the thought state of the user 20 is the deliberation state or the intuition state (see FIG. 10).

As described above, the information processing method in accordance with the present embodiment includes: an obtaining step (S101) of obtaining electroencephalogram data indicative of the electroencephalogram of the user 20 or biological information substitutable for the electroencephalogram; a first estimation step (S102) of estimating, by using the model 131 for estimating an activity state of a brain network, the activity state of the brain network from the electroencephalogram data or the biological information; and a second estimation step (S103) of estimating whether or not a thought state of the user 20 is the deliberation state, on the basis of a probability that the activity state of the brain network estimated in the first estimation step switches.

### ADVANTAGEOUS EFFECTS

As described above, according to the first embodiment, the following advantageous effects can be obtained.

The information processing device 10 comprises: an obtaining section 111 that obtains electroencephalogram data indicative of (a) an electroencephalogram of the user 20 or (b) biological information substitutable for the electroencephalogram; a first estimation section 112 that estimates, by using a model 131 for estimating an activity state of a brain network, the activity state of the brain network from the electroencephalogram data or the biological information; and a second estimation section 113 that estimates whether or not a thought state of the user is the deliberation state, on the basis of a probability that the activity state of the brain network estimated by the first estimation section 112 switches. The brain network may include at least a default mode network, a salience network, and an executive network. Furthermore, the model may include at least one selected from the group consisting of a machine learning model, a template model, and a regression model.

As described above, the development of AI in recent years may lead to the following problem. That is, as AI carries out deliberation on behalf of people, people may get used to using only intuition and become unable to activate deliberation. In order to solve such a problem, there has been a demand for development of a system that provides a stimulus to trigger activation of deliberation. For this purpose, it is first necessary to acknowledge whether a thought state is the deliberation state or the intuition state. According to the above configuration, the electroencephalogram data or the biological information substitutable for the electroencephalogram is used, which makes it possible to estimate the thought state of the user 20 in a non-invasive manner.

Furthermore, if the probability of the activity state of the brain network estimated by the first estimation section 112 switching per unit time is higher than a preset threshold, the second estimation section 113 may estimate that the thought state of the user 20 is the deliberation state. As explained with reference to FIG. 10, the “unit time” mentioned here is 2 seconds, for example.

As described above, with the configuration that uses the probability of the activity state of the brain network switching per unit time, it is possible to estimate the thought state of the user 20 with high accuracy.

### Second Embodiment

### Next, the Following Will Describe a Second

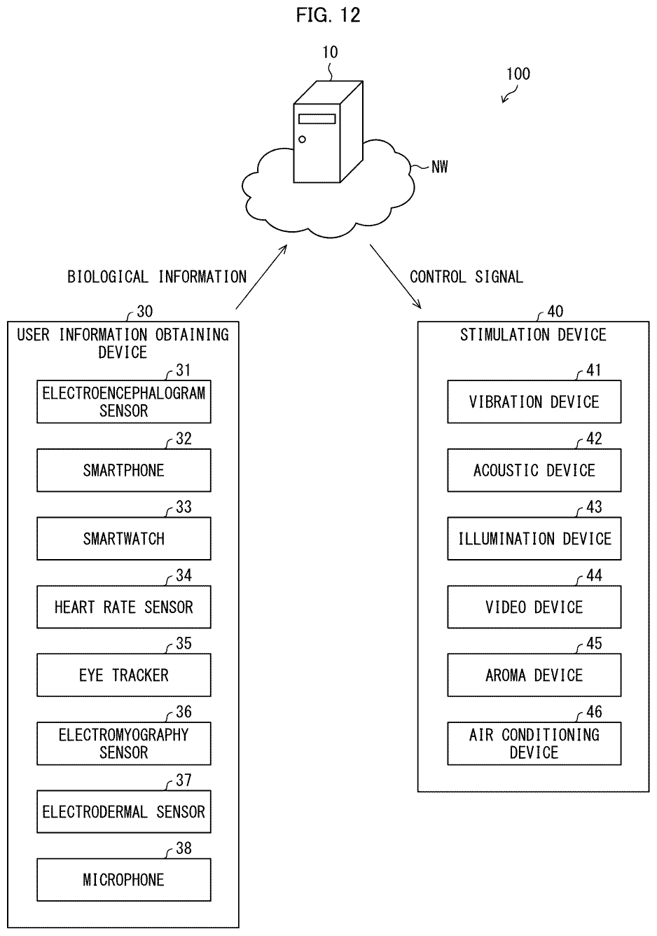

embodiment of the present disclosure. FIG. 12 is a view illustrating an example of a configuration of an information processing system 100 in accordance with a second embodiment. For features identical to those of the first embodiment, the same reference signs are used, and descriptions thereof will not be repeated. The following description will focus on differences therebetween.

As shown in FIG. 12, the information processing system 100 in accordance with the second embodiment further includes a stimulation device 40 in addition to an information processing device 10 and a user information obtaining device 30. The information processing device 10 controls the stimulation device 40 that stimulates a user 20 on the basis of an estimation result of a thought state, which has been described in the first embodiment. Specifically, the information processing device 10 generates a control signal for controlling the stimulation device 40 and transmits the generated control signal to the stimulation device 40. The stimulation device 40 operates in accordance with the control signal received from the information processing device 10, and stimulates the user 20.

In this way, the information processing system 100 in accordance with the second embodiment corresponds to the above-described “system that provides a stimulus to trigger activation of deliberation”.

### Stimulation Device 40

The stimulation device 40 is a device that stimulates the user 20's senses of vision, hearing, touch, smell, and the like and that operates in accordance with the control signal received from the information processing device 10 so as to stimulate the user 20 as described above. The stimulation device 40 includes a vibration device 41, an acoustic device 42, an illumination device 43, a video device 44, an aroma device 45, and an air conditioning device 46.

The vibration device 41 is a device that stimulates the sense of touch, an example of which is a chair with a vibrator. The acoustic device 42 is a device that stimulates the sense of hearing, an example of which is a speaker. The illumination device 43 is a device that stimulates the sense of vision, an example of which is a ceiling light. The video device 44 is a device that stimulates the sense of vision, an example of which is a television. The aroma device 45 is a device that stimulates the sense of smell, an example of which is an aroma diffuser. The air conditioning device 46 is a device that stimulates the sense of touch, an example of which is an air conditioner.

Note that the video device 44 that stimulates the sense of vision is not limited to the television. Alternatively, another device may be used as the video device 44, as long as the device operates upon receiving a control signal from an external device (e.g., the information processing device 10). For example, as the video device 44 that stimulates the sense of vision, a virtual reality (VR) headset may be used.

The vibration device 41 that stimulates the sense of touch is not limited to the chair with a vibrator. Alternatively, another device may be used, as long as the device operates upon receiving a control signal from an external device (e.g., the information processing device 10). For example, as the vibration device 41 that stimulates the sense of touch, a haptic feedback device, represented by a haptics device, may be used.

Such a stimulation device 40 may be configured integrally with the smartphone 32, provided that the stimulation device 40 can be mounted to the smartphone 32. Consequently, with a single device, it is possible to obtain the biological information and activate deliberation. Note that the terminal which can be carried by the user 20 is not limited to the smartphone 32. The biological information measurement function, the thought state estimation function, and the deliberation activation function may be mounted on a terminal other than the smartphone 32 which can be carried by the user 20.

### Functional Example of Processor 11

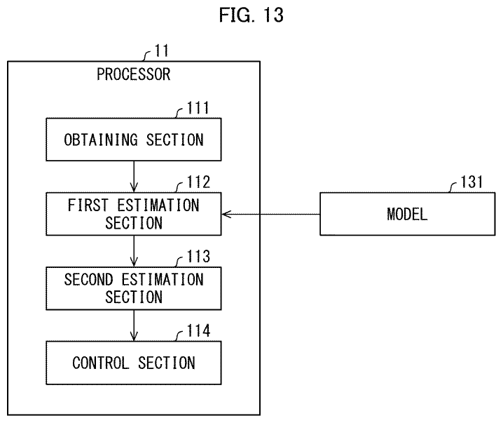

As shown in FIG. 13, the processor 11 functions as a control section 114 in addition to the obtaining section 111, the first estimation section 112, and the second estimation section 113 described in the first embodiment.

The control section 114 generates a control signal for controlling the stimulation device 40, and transmits the generated control signal to the stimulation device 40. There is no particular limitation on the method for generating the control signal. For example, the control signal may be generated by the following method. That is, the control section 114 may extract a feature(s) indicating a relation between a stimulus and switching of the activity state of the brain network and cause the feature(s) to be learned. The control section 114 causes a trained model to carry out reinforcement learning on a switching frequency of the activity state of the brain network. By using the model generated in this manner, the control section 114 may generate a control signal for generating a stimulus that guides the thought state of the user 20 to the deliberation state.

### Flow of Processes

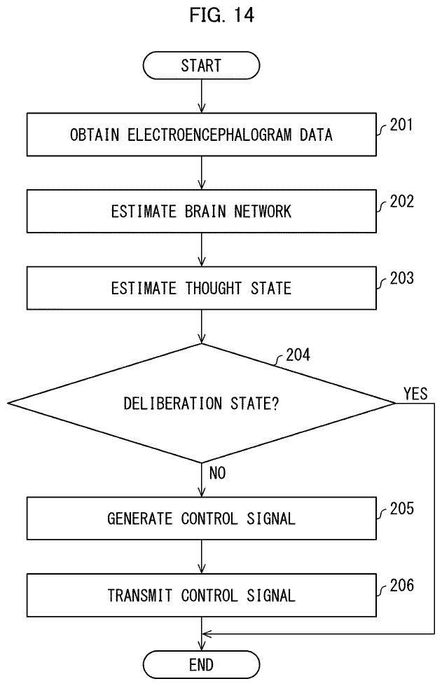

Next, the following will describe, with reference to FIG. 14, a flow of processes executed by the information processing device 10 in accordance with the second embodiment. However, since the processes of steps S201 to S203 are the same as the processes of steps S101 to S103 shown in FIG. 11, descriptions thereof will be omitted as appropriate.

Through the processes of steps S201 to S203, it is estimated whether the thought state of the user 20 is the deliberation state or the intuition state.

If it is estimated that the thought state is the deliberation state (YES in step S204), the series of processes ends. On the other hand, if it is estimated that the thought state is not the deliberation state, that is, the thought state is the intuition state (NO in step S204), the process proceeds to step S205.

In step S205, the control section 114 generates a control signal for generating, by using a model that has undergone reinforcement learning on the switching frequency of the activity state of the brain network, a stimulus that guides the thought state of the user 20 to the deliberation state.

In step S206, the control section 114 outputs the control signal generated in the process of step S205 to the stimulation device 40. The stimulation device 40 operates in accordance with the control signal, and stimulates the user 20.

As described above, if the second estimation section 113 estimates that the user 20 is not in the deliberation state, the control section 114 in accordance with the second embodiment transmits, to an external device, a signal for generating a stimulus that guides the thought state of the user 20 to the deliberation state.

With the above configuration, it is possible to provide the user 20 with a stimulus that triggers activation of deliberation. Therefore, even if AI develops further in the future, the user 20 can activate deliberation.

### Software Implementation Example

Functions of the information processing device 10 (hereinafter, simply referred to as “device”) can be realized by a program (information processing program) for causing a computer to function as the device, the program causing the computer to function as the control blocks (the obtaining section 111, the first estimation section 112, the second estimation section 113, and the control section 114) of the device.

In this case, the device includes a computer having at least one device (e.g., a processor) and at least one storage device (e.g., a memory) as hardware for executing the program. Each function described in the foregoing embodiments can be realized by the computer executing the program.

The program may be stored in one or more non-transitory computer-readable storage media. These storage media may or may not be provided in the device. In the latter case, the program may be supplied to the device via any wired or wireless transmission medium.

Moreover, some or all of functions of the control blocks can be realized by a logic circuit. For example, the present disclosure encompasses, in its scope, an integrated circuit in which a logic circuit that functions as each of the control blocks is formed. As another alternative, for example, it is possible to realize the functions of the control blocks by a quantum computer.

Furthermore, each of the processes which are described in the foregoing embodiments may be carried out by artificial intelligence (AI). In this case, the AI may be operated by the device, or alternatively operated by another device (e.g., an edge computer, a cloud server, or the like).

### SUPPLEMENTARY NOTES

The present disclosure can also be expressed as follows.

An information processing device in accordance with a first aspect of the present disclosure includes: an obtaining section that obtains (a) electroencephalogram data indicative of an electroencephalogram of a user or (b) biological information substitutable for the electroencephalogram; a first estimation section that estimates, by using a model for estimating an activity state of a brain network, the activity state of the brain network from the electroencephalogram data or the biological information; and a second estimation section that estimates whether or not a thought state of the user is a deliberation state, on a basis of a probability that the activity state of the brain network estimated by the first estimation section switches.

An information processing device in accordance with a second aspect of the present disclosure may be configured such that, in the first aspect, the second estimation section estimates that the thought state of the user is the deliberation state, in a case where the probability that the activity state of the brain network estimated by the first estimation section switches per unit time is higher than a preset threshold.

An information processing device in accordance with a third aspect of the present disclosure may be configured such that, in the first or second aspect, the brain network includes at least a default mode network, a salience network, and an executive network.

An information processing device in accordance with a fourth aspect of the present disclosure may be configured such that, in any one of the first to third aspects, the model includes at least one selected from the group consisting of a machine learning model, a template model, and a regression model.

An information processing device in accordance with a fifth aspect of the present disclosure may be configured such that, in any one of the first to fourth aspects, the information processing device further includes: a control section that transmits, in a case where the second estimation section estimates that the user is not in the deliberation state, a signal to an external device, the signal being for generating a stimulus that guides the thought state of the user to the deliberation state.

An information processing program in accordance with a sixth aspect of the present disclosure may be an information processing program causing a computer to function as the information processing device in accordance with the first aspect, the information processing program causing the computer to function as the obtaining section, the first estimation section, and the second estimation section.

An information processing method in accordance with a seventh aspect of the present disclosure is a method including: an obtaining step of obtaining (a) electroencephalogram data indicative of an electroencephalogram of a user or (b) biological information substitutable for the electroencephalogram; a first estimation step of estimating, by using a model for estimating an activity state of a brain network, the activity state of the brain network from the electroencephalogram data or the biological information; and a second estimation step of estimating whether or not a thought state of the user is a deliberation state, on a basis of a probability that the activity state of the brain network estimated in the first estimation step switches.

The present disclosure is not limited to the above-described embodiments, but can be altered by a skilled person in the art within the scope of the claims. The present disclosure also encompasses, in its technical scope of the present disclosure, any embodiment derived by combining technical means disclosed in differing embodiments.

### REFERENCE SIGNS LIST

100: information processing system
10: information processing device
30: user information obtaining device
31: electroencephalogram sensor
32: smartphone
33: smartwatch
34: heart rate sensor
35: eye tracker
36: electromyography sensor
37: electrodermal sensor
38: microphone
40: stimulation device
41: vibration device
42: acoustic device
43: illumination device
44: video device
45: aroma device
46: air conditioning device
111: obtaining section
112: first estimation section
113: second estimation section
114: control section
131: model

1. An information processing device, comprising:
an obtaining section that obtains (a) electroencephalogram data indicative of an electroencephalogram of a user or (b) biological information substitutable for the electroencephalogram;
a first estimation section that estimates, by using a model for estimating an activity state of a brain network, the activity state of the brain network from the electroencephalogram data or the biological information; and
a second estimation section that estimates whether or not a thought state of the user is a deliberation state, on a basis of a probability that the activity state of the brain network estimated by the first estimation section switches.

2. The information processing device according to claim 1, wherein:
the second estimation section estimates that the thought state of the user is the deliberation state, in a case where the probability that the activity state of the brain network estimated by the first estimation section switches per unit time is higher than a preset threshold.

3. The information processing device according to claim 1, wherein:
the brain network includes at least a default mode network, a salience network, and an executive network.

4. The information processing device according to claim 1, wherein:
the model includes at least one selected from the group consisting of a machine learning model, a template model, and a regression model.

5. The information processing device according to claim 1, further comprising:
a control section that transmits, in a case where the second estimation section estimates that the user is not in the deliberation state, a signal to an external device, the signal being for generating a stimulus that guides the thought state of the user to the deliberation state.

6. A non-transitory computer-readable storage medium in which an information processing program is stored, the information processing program causing a computer to function as an information processing device recited in claim 1, the information processing program causing the computer to function as the obtaining section, the first estimation section, and the second estimation section.

7. An information processing method, comprising:
obtaining (a) electroencephalogram data indicative of an electroencephalogram of a user or (b) biological information substitutable for the electroencephalogram;
estimating, by using a model for estimating an activity state of a brain network, the activity state of the brain network from the electroencephalogram data or the biological information; and
estimating whether or not a thought state of the user is a deliberation state, on a basis of a probability that the activity state of the brain network estimated switches.
